(19) United States
(12) Patent Application Publication
(10) Pub. No.: US 2024/0366630 A1
(43) Pub. Date: Nov. 7, 2024

(54) Treatment to infective illnesses 2

(71) Applicant: Menni Menashe Zinger, Ashkelon (IL)

(72) Inventor: Menni Menashe Zinger, Ashkelon (IL)

(21) Appl. No.: 18/144,181

(22) Filed: May 6, 2023

(51) Int. Cl. A61K 31/568; A61K 9/00; A61K 31/198

(52) U.S. Cl. CPC A61K 31/568; A61K 9/0014; A61K 31/198

(57) ABSTRACT

At this invention I present medication treatment vaccine to smallpox and maybe also to the plague and ebola and other infective illnesses.

Not applicable

### DESCRIPTION

At man at his 40^{th}, using arginine/testosterone caused pimple with black color to disappear at 4-5 days using shampoo with arginine which is raising the level of testosterone at body each time from his feet/finger.

1. using shampoo with arginine/testosterone as medication treatment/vaccine/food supplement at shampoo/soap/foam/tablet/pill/capsules/injection/s powder/patch, in order to treat/cure small pox/the plague/ebola and even after 4-5 times of using this shampoo in order to wash the hair on head, there can be significant improvement even 100% without pimples.
